(19) United States
(12) Patent Application Publication
(10) Pub. No.: US 2015/0346201 A1
(43) Pub. Date: Dec. 3, 2015

(54) SYSTEM AND METHOD FOR PICOLITER VOLUME MICROFLUIDIC DIAGNOSTICS

(71) Applicants: Tali KORNY, (US); Alexander GOLDBERG, (US); Martin L. YARMUSH, (US)

(72) Inventors: Tali Korny, Boston, MA (US); Alexander Goldberg, Boston, MA (US); Martin L. Yarmush, Newton, MA (US)

(21) Appl. No.: 14/759,267

(22) PCT Filed: Jan. 7, 2014
(86) PCT No.: PCT/US2014/010412

§ 371 Date: Jul. 6, 2015

Related U.S. Application Data
(60) Provisional application No. 61/749,733, filed on Jan. 7, 2013.
(60) Provisional application No. 61/777,173, filed on Mar. 12, 2013.

(51) Int. Cl. G01N 33/569; G01N 21/64; G01N 33/543

(52) U.S. Cl. CPC G01N 33/56916; G01N 33/54306; G01N 21/6428; G01N 2201/062

(57) ABSTRACT

The present invention provides a method for a picoliter volume microfluidic assay. The method includes the steps of providing a first solution comprising a sensor, preparing a sample comprising at least one analyte, and combining the sample with an indicator, thereby forming a second solution. The method further includes co-encapsulating the first solution and the second solution in a plurality of droplets with a microfluidic device, incubating the plurality of droplets, thereby forming at least one complex comprising the sensor, at least one analyte and indicator, and detecting the at least one complex. A primary signal associated with the at least one complex is distinguishable from a background signal associated with at least one of the sensor, at least one analyte and indicator individually.

### CROSS-REFERENCE TO RELATED APPLICATIONS

This application is based on, claims the benefit of, and incorporates herein by reference U.S. Provisional Application No. 61/749,733, filed Jan. 7, 2013, and U.S. Provisional Application No. 61/777,173, filed Mar. 12, 2013.

### STATEMENT REGARDING FEDERALLY SPONSORED RESEARCH

This invention was made with government support under grant no. CA174401 awarded by the National Institutes of Health. The government has certain rights in this invention.

### BACKGROUND OF THE INVENTION

The disclosure relates, in general, to small volume diagnostic assays and, more particularly, to picoliter volume microfluidic immunodetection assays for point-of-care diagnostics.

Point-of-care (POC) diagnostics have attracted much interest and investment for their promise to transform the global health. Emerging POC applications have already improved the health care system with respect to particular applications, such as infectious diseases diagnostics including syphilis, dengue and HIV. Nevertheless, multiple barriers still exist for worldwide acceptance of the POC approach. Today, enzyme-linked immunosorbent assay (ELISA) is the gold standard in the POC diagnostics for evaluation of biomarkers and detection of bio-analytes because of its sensitivity, accuracy and repeatability. Although widely used in clinical and basic biomedical research, standard ELISA diagnostics consume significant labor, time, volumes of analyte and expensive reagents (>10 μL) per each reaction step. To reduce reagent volumes, a multiplex cytometric immunosorbent microsphere based array was introduced (Leng et al., 2006. J Gerontol A Biol Sci Med Sci 63(8):879-884). However, the device cost, complex laser-based equipment, and an advanced operator skill set prevent worldwide practice of the microsphere-multiplex immunosorbent technique in POC diagnostics to date. Therefore, a need exists for low cost, fast and sensitive techniques to analyze bio-analytes for global POC diagnostics.

In the last decades, microelectromechanical systems (MEMS) reduced the total bio-assaying costs by miniaturization of laboratory equipment and volumes of reagents used. Droplet microfluidics is an emerging MEMS technique with the potential to revolutionize the miniaturization of bioassays. Droplet microfluidic devices use two phase system of water droplets formed in oil to reduce reaction volume, isolate and separate reaction, prevent absorption and evaporation, and provide temperature and gas permeability control. Physical parameters of droplets including size, formation frequency, flow rate and mixing, are controlled by the geometry of the microfluidic channel, oil composition and fluids flow rates. Moreover, the flow of water droplets in the oil medium generates internal streamlines cause chaotic mixing, which presumably accelerates the reaction rates.

Droplet based MEMS have been successfully applied in areas such as digital polymerase chain reaction (PCR), synthetic biology and cell detection. For digital PCR, compartmentalization of reagents and effective mixing significantly increases amplification efficiency. In synthetic biology, small reaction volumes and a high-throughput design enables fast screening of multiple engineered biological parts. When assaying cells and microorganisms, droplets provide a closed, controlled environment that prevents the diffusion of the molecules produced by cells thus enabling characterization of biochemical processing on a single-cell level. Such a system was previously reported for the detection of a single T cell secretion of IL-10 cytokine (Konry et al., 2011. Biosens Bioelectron 26(5):2707-2710).

The potential of MEMS for cell detection also has applications in monitoring water quality. Worldwide water-associated infectious diseases are a major cause of morbidity and mortality. It is estimated that 4.0% of global deaths and 5.7% of the global disease burden are caused by waterborne diseases. Common waterborne diseases include diarrhea (bacterial, viral and parasitic), schistosomiasis, trachoma, ascariasis, and trichuriasis. Low income countries are particularly vulnerable to waterborne diseases because of their under-developed infrastructure and poor water management. Water and sewage distribution systems in high income societies also require pollutant and microorganism monitoring.

Escherichia coli, found in mammalian feces, has been a biological indicator for water quality since the 19th century. Testing for the presence of E. coli is obligatory for current water management systems. The identification of bacteria in a water sample includes the capture of target bacteria from the water sample, and the identification of the captured bacteria. Traditional methods for E. coli detection include culture, fermentation, and enzyme-linked immunosorbent (ELISA) and polymerase chain reaction (PCR) assays. These traditional methods have disadvantages including long identification times (2-4 days), and/or high labor and reagent costs. Despite high costs, rapid tests are necessary to enable quick responses to putative contamination threats. Recently, novel sensors and assays for rapid pathogen detection have been developed. However, many of these newer methods remain expensive and/or require sophisticated instrumentation, and most have yet to reach the market place. Therefore, there remains a need for alternative platforms for the detection of bacteria in water samples. In particular, there is a need for a system and method to inexpensively perform water quality control testing at multiple locations along a distribution system, and to rapidly process and share the test results. More generally, given the aforementioned disadvantages of current POC diagnostics and the potential of droplet MEMS technology, there is a need to develop low cost, easy to use assays for POC diagnostics.

### SUMMARY OF THE INVENTION

The present invention overcomes the aforementioned drawbacks by providing a sensitive, specific, rapid and low cost droplet microfluidics based technology for bio-analyte detection and quantification with potential applications for the POC diagnostics. In one aspect, the present disclosure provides a sensitive, specific, rapid and low cost picoliter microsphere-based platform for bioanalyte detection and quantification. In one example method, a biological sample, biosensing microspheres, and fluorescently labeled detection (secondary) antibodies are co-encapsulated to capture an analyte. (e.g., human anti-tetanus immunoglobulin G) on the surface of the microsphere in microfluidic picoliter-sized droplets. The absorption of the analyte and detecting antibodies on the microsphere concentrate the fluorescent signal in correlation with analyte concentration. Using the platform and commercially available antibodies, we were able to quantify anti-tetanus antibodies in human serum. In comparison to standard bulk immunosorbent assays, the microfluidic droplet platform presented here reduces the reagent volume by four orders of magnitude, while fast reagent mixing reduces the detection time from hours to minutes. We consider this platform to be a major leap forward in the miniaturization of immunosorbent assays and to provide a rapid and low cost tool for global point-of-care.

In another aspect, the present disclosure provides a system and method that leverage the potential of automated microscale systems for water quality analysis. To detect E. coli in water samples, a bead-based immuno-assay performed with a droplet microfluidic device to reduce reagent volume and enhance reaction rates was developed and demonstrated. The microfluidic assay was integrated with a portable imaging system and remote control automation software. Furthermore, the system capabilities were demonstrated through the detection of model coliform bacteria, E. coli, in feces-contaminated drinking water. Successful multiplex detection assay results suggest that simultaneous multiple bacteria detection, using several types of beads conjugated with different antibodies that bind different target bacteria, will be possible with further development. The present platform decreased reagent volumes, (the full chip uses 520 nL of reagents, while conventional assay require at least 10 μL of reagents) and allows for results within 8 hours from the time of water sampling. Moreover, the results demonstrate that a combination of droplet microfluidics with low cost optics and cloud network can provide a flexible and efficient alternative for pathogen detection in drinking water. The present platform has the potential to significantly improve water diagnostics, particularly in low income countries where the infrastructure does not yet exist.

In accordance with one aspect of the present disclosure, a method is provided for a picoliter volume microfluidic assay. The method includes the steps of providing a first solution comprising a sensor, preparing a sample comprising at least one analyte, and combining the sample with an indicator, thereby forming a second solution. The method further includes co-encapsulating the first solution and the second solution in a plurality of droplets with a microfluidic device, incubating the plurality of droplets, thereby forming at least one complex comprising the sensor, at least one analyte and indicator, and detecting the at least one complex. A primary signal associated with the at least one complex is distinguishable from a background signal associated with at least one of the sensor, at least one analyte and indicator individually.

In one aspect, the volume of each of the plurality of droplets is less than about 10 μL, preferably less than about 1 μL, and more preferably about 500 pL. In another aspect, the size of each of the plurality of droplets is about 100 μm. In yet another aspect, the step of co-encapsulating further comprises passing the first and second solutions through a nozzle of the microfluidic device. In still another aspect, the method further comprises arraying the plurality of droplets into a set of wells within the microfluidic device.

In one aspect, the sensor comprises a microsphere. In another aspect, the indicator comprises a fluorophore, and in yet another aspect, the at least one analyte comprises a biological sample.

In accordance with another aspect of the present disclosure, a system for performing a picoliter volume microfluidic assay is provided. The system includes a microfluidic device comprising a first solution source, a second solution source, a nozzle in communication with the first and second solution sources, the nozzle configured for co-encapsulating the first solution and the second solution in a plurality of droplets within the microfluidic device, and an array of wells in communication with the nozzle, the array of wells configured to receive the plurality of droplets. The system further includes a detector configured to detect a primary signal from at least one complex within at least one of the plurality of droplets. The at least one complex comprises a sensor, at least one analyte and an indicator, and a primary signal associated with the at least one complex is distinguishable from a background signal associated with at least one of the sensor, at least one analyte and indicator individually.

In one aspect the volume of each of the plurality of droplets is less than about 10 μL, preferably less than about 1 μL and more preferably, about 500 pL. In another aspect, the size of each of the plurality of droplets is about 100 μm. In yet another aspect, the detector is a fluorescence microscope, the sensor comprises a microsphere and the indicator comprises fluorophore. In one aspect, the at least one analyte comprises a biological sample, and in another aspect, the biological sample comprises an antibody.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic illustration of a droplet microfluidics device according to the present disclosure.

FIG. 1A is a photograph of a prototype of the droplet microfluidics device of FIG. 1.

FIG. 2. is a schematic illustration of assay components encapsulated within a droplet for an example immunosorbent reaction scheme including solution with reagents, detecting microspheres, tetanus toxoid, human anti-tetanus IgG analyte, secondary antihuman IgG antibody, and fluorophore.

FIG. 3 is a schematic illustration of absorption controlled ELISA in the well mixed solution inside the flowing droplet.

FIG. 4 is a schematic illustration of a Diffusion controlled ELISA reaction scheme

FIG. 5 is a schematic illustration of a fluid flow profile in the moving nano-litter droplet reactor.

FIG. 6 is a microscopic image of the droplet generation nozzle of the prototype of FIG. 2.

FIG. 7 is a microscopic image of the droplet detecting array of the prototype of FIG. 2.

FIG. 8 is a microscopic image of a droplet with coencapsulated sensor, analyte and indicator. Arrows indicate sensor-analyte-indicator complexes.

FIG. 9 is a phase microscopy image of the central mixing channel and nozzle of the device of FIG. 1, where tetanus toxoid conjugated microspheres (white arrow with no outline) and analyte and secondary Alexa Fluor®488 fluorescent labeled antibody (white arrow with black outline) are mixed (2.5× magnification).

FIG. 10 is a fluorescent microscopy image of the area of the device as shown in FIG. 9

FIG. 11 is a fluorescent microscopy image of the droplet generating nozzle (white arrow indicates the nozzle for droplet generation) with reagents mixture (2.5× magnification).

FIG. 12 is a fluorescent microscopy image of droplet detecting array according to FIG. 1 with the mixed reagents and microspheres.

FIG. 13 is a fluorescent microscopy image showing Human anti-tetanus IgG detection with microspheres coencapsulated in droplets. The emission signal was detected 15 min after the encapsulation and is shown at 10× magnification.

FIG. 14 is a three dimensional plot of fluorescent emission signal as a function of position along the surface of a droplet array of a platform according to the present invention.

FIG. 15 is a three dimensional fluorescent emission signal analyses for a single droplet from FIG. 14.

FIG. 16 is a calibration curve for human anti-tetanus IgG detection showing the signal-to-noise ratio (S/N) as a function of IU ml⁻¹. Error bars show ±one standard deviation of the mean.

FIG. 17 is a plot of experimental fluorescence signal as a function of standard normal quantities indicating human sample fluorescent readout distribution testing the hypothesis that the measured values have a Normal Distribution profile. R=0.9924.

FIG. 18 is a schematic illustration of a bacteria capturing and detection assay. Magnetic bead capture of E. coli from enriched water samples, and downstream chip encapsulation for fluorescent labeling and detection.

FIG. 19 is schematic illustration of a portable fluorescent optical system for signal detection and sharing.

FIG. 19A is a photograph of a prototype of the portable fluorescent optical system of FIG. 19.

FIG. 20 is a schematic illustration of a multiplex cloud-based water quality assessment system. The detector network is enabled by script-based programming language and cloud-based data storage. Users send requests for water quality assessment at different locations in the distribution system. The detection device performs the tests, the results are stored in the cloud, and the collected data is shared between users and applications.

FIG. 21 is a droplet microfluidic device as viewed from the top camera of the optical system of FIG. 19. Arrows indicate tubing/chip connection locations as follows: o, oil (inlet); b, beads conjugated with E. coli (inlet); a, fluorescently labeled antibody (inlet); w, waste (outlets).

FIG. 22 is a microscopic image of a droplet as seen from the top camera of the optical system of FIG. 19 with white LED illumination.

FIG. 23 is a microscopic image of antibody green fluorescence indicative of the presence of E. coli (positive control).

FIG. 24 is a photograph showing gel electrophoresis analysis of PCR reaction products for four contaminated samples, along with positive and negative controls for contaminated water assays. Gel lanes correspond to the amplification of loci as follows: lane 1, 16S rRNA primary locus; lane 2, 16S rRNA secondary locus; lane 3, tuf; lane 4, uidA.

FIG. 25 is a schematic overview of a method for operation of a cloud-based water quality assessment system. The optical system and device are integrated with by a script based programming language and cloud-based data storage. Users send requests for water quality assessment at different locations in the distribution system and the device and optical system perform the tests. Results are stored in the cloud, and the collected data is shared between users and applications.

FIG. 26 shows a representative microscopic fluorescence image demonstrating fecal E. coli detection in drinking water (white arrows).

### DETAILED DESCRIPTION OF THE INVENTION

The present invention is presented in several varying embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the system. One skilled in the relevant art will recognize, however, that the system and method may both be practiced without one or more of the specific details, or with other methods, components, materials, and so forth. In other instances, well-known structures, materials, or operations are not shown or described in detail to avoid obscuring aspects of the invention.

The present disclosure provides a picoliter volume microfluidic system for the detection of one or more target analytes. First, a physical model of immuno-absorbent reaction kinetics was developed to describe the system. Next, the system was applied to the detection of an analyte in human serum-human anti-tetanus IgG. Finally, the system was tested in a multiplex format in combination with remote automation and cloud-based script execution and data storage for the detection of contaminants in drinking water.

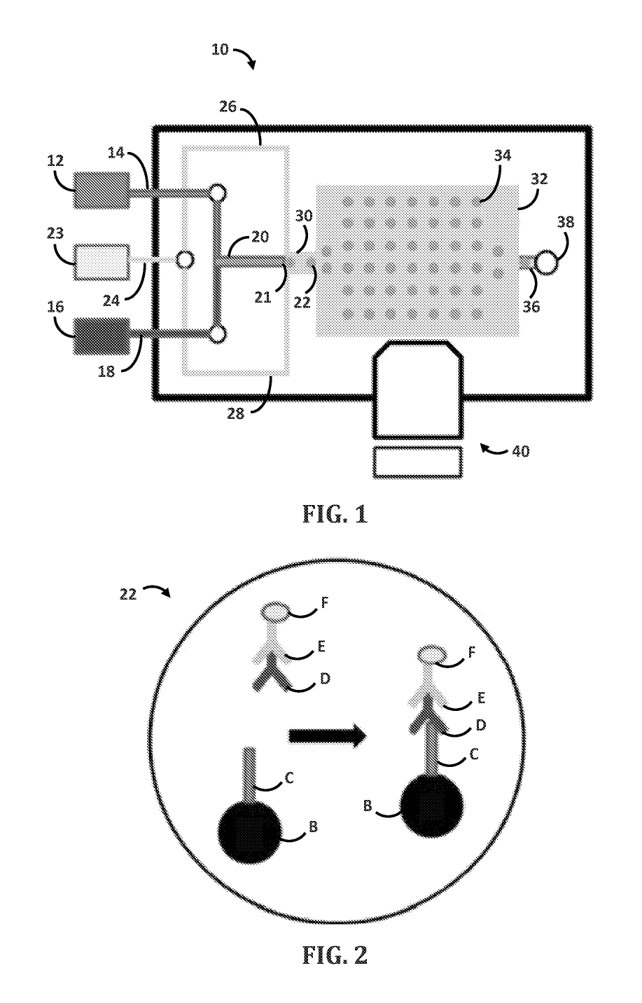

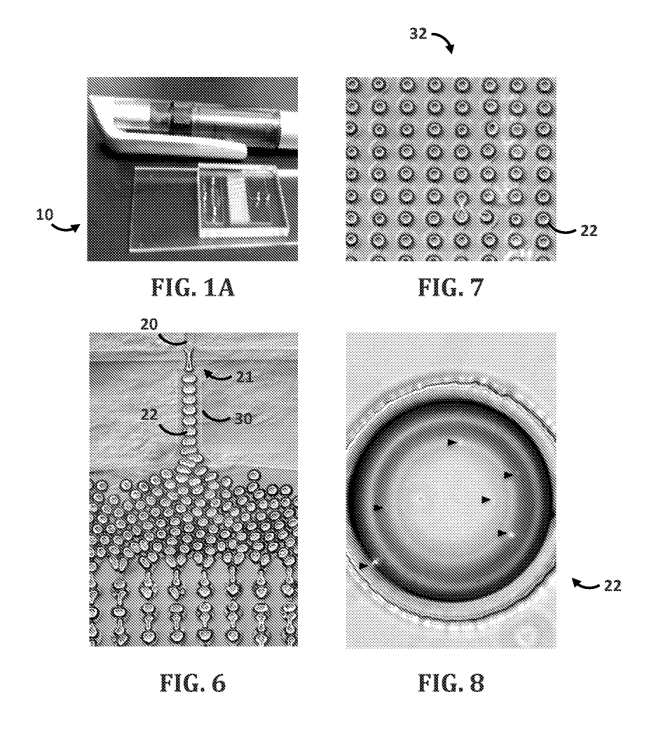

An example of a microfluidic platform 10 according to the present disclosure is schematically illustrated in FIG. 1. This basic reagents encapsulation device 10 uses a flow focusing geometry to produce aqueous droplets suspended in an organic (e.g., oil) phase (Konry et al., 2011, Biosensors and Bioelectronics 26(5), 2707-2710; Goldberg et al., 2013, Microchim Acta 180:855-860). The device 10 can accommodate at least one aqueous stream and at least one organic stream. In the present example, a first aqueous pump 12 is configured to provide a first aqueous solution to a first inlet channel 14. In addition, a second aqueous pump 16 is configured to provide a second aqueous solution to a second inlet channel 18. The first and second inlet channels 14, 18 are in communication with a central mixing conduit 20, wherein the first and second aqueous solutions are admixed. The central mixing conduit 20 terminates at a droplet forming nozzle 21 for coencapsulating the first and second aqueous solution admixture in picoliter-scale droplets 22.

The device 10 further includes an organic phase pump 23 configured to provide an organic solution to an inlet channel 24. The inlet channel 24 is in fluid communication with a first organic conduit 26 and a second organic conduit 28. The first and second organic conduits 26, 28 converge at the outlet of the nozzle 21 in order to encapsulate the aqueous droplets formed by the nozzle 21 in the organic phase. The outlet of the nozzle 21 and the first and second organic conduits 26, 28 are in communication with passage 30. Passage 30 is in communication with a detecting array 32 that includes a number of wells 34 for capturing the droplets 22 in defined locations. Material flowing through the device 10, including the organic phase, exits the detecting array 32 via an outlet passage 36 in communication with an outlet 38. FIG. 1 also illustrates a detector in the form of a microscope objective 40 for analyzing the arrayed droplets 22 in the wells 34 and for detecting a signal such as a fluorescence signal associated with the droplets 22.

In order to better understand and refine the present system and methods, a physical model of immuno-absorbent reaction kinetics was developed. In particular, physical models can be used to describe reaction kinetics relevant to immune-absorption, standard well-based ELISA, microspot assays and the system of the present disclosure. The foundation of the model comprises general laws governing the reaction kinetics, and the equations governing the reaction in classical well-based ELISA. Based on this foundation, the set of equations that govern the reaction kinetics inside moving droplets in the present system was developed.

In general, the model includes governing equations for mass transport and surface absorption. The general conservation equation for the reaction in the fluid bulk appears in Eq. 1:

$$\frac{\partial C}{\partial t} + ▽ ( - D ▽ C + vC ) = {R}_{v} \qquad ( Eq . 1 )$$

where C is the bulk concentration of an analyte, D is the bulk diffusivity analyte, and v is the fully developed velocity profile of an analyte in the bulk. Rᵥ is the volumetric rate of analyte creation. The initial condition for the analyte concentration in the bulk is given by Eq. 2:

C(t=0)=C₀ (Eq. 2)

The conservation equation for the detecting surface that includes the surface diffusion and the reaction rate for the formation of the absorbed analyte is given by Eq. 3:

$$\frac{\partial {C}_{s}}{\partial t} + ▽ ( - {D}_{s} ▽ {C}_{s} ) = {k}_{on} {C}_{( n = 0 )} ( {C}_{s 0} - {C}_{s} ) - {k}_{off} {C}_{s} \qquad ( Eq . 3 )$$

where Cₛ is the surface concentration of an analyte, Dₛ is the analyte surface diffusivity Cₙ₌₀ is the concentration of bulk analyte near the reactive bead wall, Cₛ₀ is the total number of the biding sites, kₒₙ is the association rate constant and k_{off} is the dissociation rate constant of the binding reaction. The initial condition for Eq. 3 is that the concentration of the absorbed species on the detecting surface at the beginning of the process is 0.

C_{S}(t=0)=0 (Eq. 4)

Equations 1 and 3 are coupled through the flux balance boundary condition on the reacting surface as follows:

n(−D∇C+vC)=−kₒₙC₍ₙ₌₀₎(Cₛ₀−Cₛ)−k_{off}C_{S} (Eq. 5)

where n is the surface vector. The additional boundary condition on non-reactant surfaces is insulation:

n(−Dₛ∇Cₛ)=0 (Eq. 6)

In order to describe the immuno-absorbent reaction kinetics for anti-tetanus IgG detection, a further system of equations was developed. FIG. 2 describes the basic chemical reaction of the anti-tetanus IgG immune absorbent reaction in the control volume of an example droplet 22. The basic components for the detection of anti-tetanus IgG in the assay are: microsphere detecting surface (B), conjugated tetanus toxoid (C), human anti-tetanus IgG analyte (D) and secondary anti-human IgG antibody (E) with the conjugated fluorophore (F). In the present example assay, the tetanus toxoid is conjugated to the bead (B-C sensor complex) and the fluorophore labeled antibody (indicator) is conjugated with the analyte (D-E-F complex) before the surface absorbance reaction. Therefore, the final absorption/dissociation reaction detected in this assay is described by Eq. 7:

$$B - C + D - E - F {{⇔}^{{k}_{on}}}_{{k}_{off}} B - C - D - E - F \qquad ( Eq . 7 )$$

where the B-C-D-E-F complex concentration is proportional to the signal emitted by the fluorophore molecule, kₒₙ is the absorption kinetic constant and k_{off} is the dissociation kinetic constant.

In the terms of the Eqs. 1-6:

C_{S}=[B−C−D−E−F] (Eq. 8a)

C=[D−E−F] (Eq. 8b)

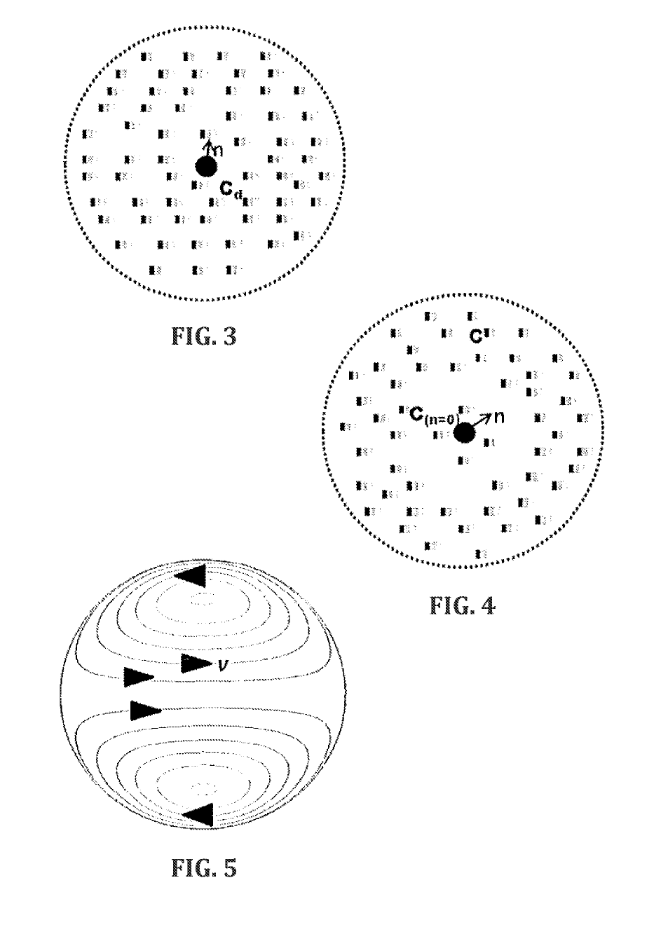

A mathematical model that was previously developed by Kankare and Vinokurov describes reaction kinetics in standard, non-mixed immunosorbent reactions on spherical surfaces (Kankare, et al., 1999, Langmuir 15(17), 5591-5599). To solve the general conservation equation for the reaction in the fluid bulk (Eqs. 1-6), a number of assumptions were made. First, it was assumed that the reaction takes place in the absence of the agitating solution, and therefore, v=0. Second, no reagents are assumed to be formed in the bulk, and therefore, Rᵥ=0. Third, it was also assumed that there was no surface diffusivity of the absorbed analyte, and thus, Dₛ=0. Fourth, and finally, the assumption of constant diffusivity of the analyte in the liquid (D is constant) was made. To compare reaction kinetics in present platform to standard non-mixed immunosorbent assays on bead, Kankare and Vinokurov model was applied to calculate the reaction time (tₕ) on bead in the not agitating solution. A calculation of tₕ was made for the present platform (FIG. 3, Eqs. 1-6) and compared to the tₕ of non-mixed bead-based immunosorbent assays (FIG. 4). Equations 9-13 describe the absorption of an analyte on the spherical bead in the non-mixed immunosorbent reaction (FIG. 4):

$$\frac{\partial C}{\partial t} - D \frac{{d}^{2}}{d {n}^{2}} - \frac{2 D}{n + R} \frac{\partial c}{\partial n} = 0 \qquad ( Eq . 9 )$$

where R is the detection bead radius.

The initial condition for the analyte concentration in the bulk is given by Eq. 10:

C(t=0)=C₀ (Eq. 10)

The surface reaction is given by Eq. 11:

$$\frac{\partial {C}_{s}}{\partial t} = {k}_{on} {C}_{( n = R )} ( {C}_{s 0} - {C}_{s} ) - {k}_{off} {C}_{s} \qquad ( Eq . 11 )$$

with initial conditions given by Eq. 12:

C_{S}(t=0)=0 (Eq. 12)

Finally, the coupling boundary condition is given by Eq. 13:

n(−D∇C)=−kₒₙC₍ₙ₌₀₎(Cₛ₀−Cₛ)−k_{off}C_{S} (Eq. 13)

The numerical solution of this set of equations showed that the time to achieve full surface coverage of the analyte on the bead surface (equilibrium) in non-mixed solution was infinity. One outcome of the numerical solution for non-mixed solutions is an approximation of the time needed for the reaction to achieve a certain deviation from the equilibrium coverage (h) on the spherical bead:

$${lim}_{{k}_{on} -> ∞} {t}_{h} \approx \frac{{Rk}_{on} {C}_{s 0}}{{k}_{off} {D ( 1 + \frac{{k}_{on}}{{k}_{off}} {C}_{0} )}^{2}} ln h \qquad ( Eq . 14 )$$

where h is the deviation from the equilibrium coverage as defined by Eq. 15:

$$h = \frac{{C}_{s 0} - {C}_{s}}{{C}_{s 0}} \qquad ( Eq . 15 )$$

With respect to reaction kinetics in droplet-based reactions, the fluid flow profile in the droplets allows constant mixing of the reagents in the cavity and therefore is different from reaction kinetics in non-agitating solutions. FIG. 5 illustrates the fluid streamlines inside the moving spherical droplet. The type of mixing shown schematically on FIG. 5 can be inefficient since the recirculation zones are hydrodynamically isolated from each other and a microfluidic wave channel geometry allow to mix reagents in moving droplets in less than 10 ms. Chaotic convection mixing process takes place inside the moving droplet reactors with biochemical reactions, as it was demonstrated previously both by analytically and imaging systems. It is therefore, in the mixed droplet the reaction rate is defined by both convection and diffusion. The convection time scale inside the ideal droplet is given by Eq. 16:

t_{conv}≈2r/v_{d} (Eq. 16)

where v_{d} is the velocity of the moving droplet, and r is the droplet radius. The diffusion time scale is given by Eq. 17:

t_{dif}≈r²/(π²D) (Eq. 17)

Table 1 provides values of parameters associated with an example device 10 according to the present system and method. Based on Eqs. 16 and 17 and the values in Table 1, both diffusion time and convection time for the reaction in the droplet were calculated. It was determined that t_{dif} was about 16 seconds, t_{conv} was about 1.5*10⁻⁴ seconds (at passage 30, FIG. 1) and t_{conv} was about 10⁻³ seconds (at detecting array 32 in FIG. 1). The convection time scale is about four to five orders of magnitude higher than the diffusion time scale and therefore, according to the present calculations, convection is the dominating mixing process for reaction inside nano-liter droplet reactor.

TABLE 1

| Parameter | Value | | Reference |
|---|---|---|---|
| Flow Rate | 30 | μL min⁻¹ | |
| Channel cross section first | 6.25 * 10⁻⁸ | m² | FIGS. 1 and 6 |
| 1 mm of flow | | | |
| Channel cross section second | 3.13 * 10⁻⁷ | m² | FIGS. 1 and 7 |
| 4 mm of flow | | | |
| Analyte bulk diffusivity (D) | 6 * 10⁻⁷ cm² | s⁻¹ | |

The reaction kinetics for a platform of according to the present system and method can vary from those associated with a non-agitated platform. In one aspect of the present system, the beads constantly move within the droplet creating irregularity, which possibly breaks the perfect recirculation zones of the ideal chaotic mixing profile. The fast mixing profile of the reaction reagents in the picoliter droplet reactors implies that the absorption reaction on the detecting beads takes places in the well-mixed solution. Thus, the present system differs from classical non-mixed bulk immune-absorbent assays such as ELISA or microspot array assays. Equations 18-21 describe detection reactions inside the mixing droplet:

$$\frac{\partial {C}_{s}}{\partial t} = {k}_{on} {C}_{d} ( {C}_{s 0} - {C}_{s} ) - {k}_{off} {C}_{s} \qquad ( Eq . 18 )$$

with the initial condition:

C_{S}(t=0)=0 (Eq. 19)

where C_{d} is the analyte concentration the whole droplet bulk volume. The full solution to Eqs. 18 and 19 appears in Eq. 20.

$${C}_{s} = - \frac{{k}_{on} {C}_{d} {C}_{s 0}}{{k}_{on} {C}_{d} + {k}_{off}} {e}^{- ( {k}_{on} {C}_{d} + {k}_{off} ) t} + \frac{{k}_{on} {C}_{d} {C}_{s 0}}{{k}_{on} {C}_{d} + {k}_{off}} \qquad ( Eq . 20 )$$

The time tₕ for reaction in the well mixed nanoliter droplet for a deviation h from the equilibrium coverage is:

$${t}_{h} = - \frac{1}{{k}_{on} {C}_{d} + {k}_{off}} ln ( h ( 1 + \frac{{k}_{off}}{{k}_{on} {C}_{d}} ) - \frac{{k}_{off}}{{k}_{on} {C}_{d}} ) \qquad ( Eq . 21 )$$

or, for reactions with small binding constants:

$${lim}_{{k}_{on} -> ∞} {t}_{h} \approx - ln ( h \text{/} {k}_{on} ) \approx 0 \qquad ( Eq . 22 )$$

In one aspect, analysis of the equations shows that for immunosorbent reactions with small binding constants (kₒₙ→∞) the time to achieve coverage h of the bead in nano-volume reactor is faster than in the bulk or micro spot array.

Following the theoretical analyses of reaction kinetics, an experimental microfluidic platform for rapid anti-tetanus IgG detection was developed. An example platform according to the present disclosure was used to encapsulate the analyte and assay reagents along with beads previously conjugated to tetanus toxin. Referring again to the device 10 of FIG. 1, in operation, individual pumps 12, 16, 23 are used to control flow rates of the organic and aqueous phases. In one specific example, pump 12 provides a first aqueous solution containing sensor beads (i.e., microspheres with conjugated tetanus toxoid, reagents B-C FIG. 2) at a concentration of 0.5 mg ml⁻¹. Concurrently, pump 16 provides the second aqueous solution containing an analyte and an indicator (i.e., D-E-F conjugate) as described in FIG. 2. The first and second solutions are passed to the central mixing conduit 20 to provide a mixture of sensor, analyte and indicator, while the first and second organic conduits 26, 28 contain the oil phase provided by pump 23. FIG. 6 shows an example T-shaped droplet generator at 2.5× magnification, while FIG. 7 shows part of an example droplet detection array, also at 2.5× magnification. FIG. 8 shows at 20× magnification the droplet with detecting beads in the array. To form droplets, the ratio of the flow rate of the aqueous admixture in the central conduit 20 to the organic phase is adjusted to 1, thereby generating a droplet volume of about 520 pL, which corresponds to a spherical drop diameter of about 100 μm. In comparison, standard ELISA kits for anti tetanus antibody detection use about 100 μL (10⁸ pL) of analyte and reagents per each reaction well.

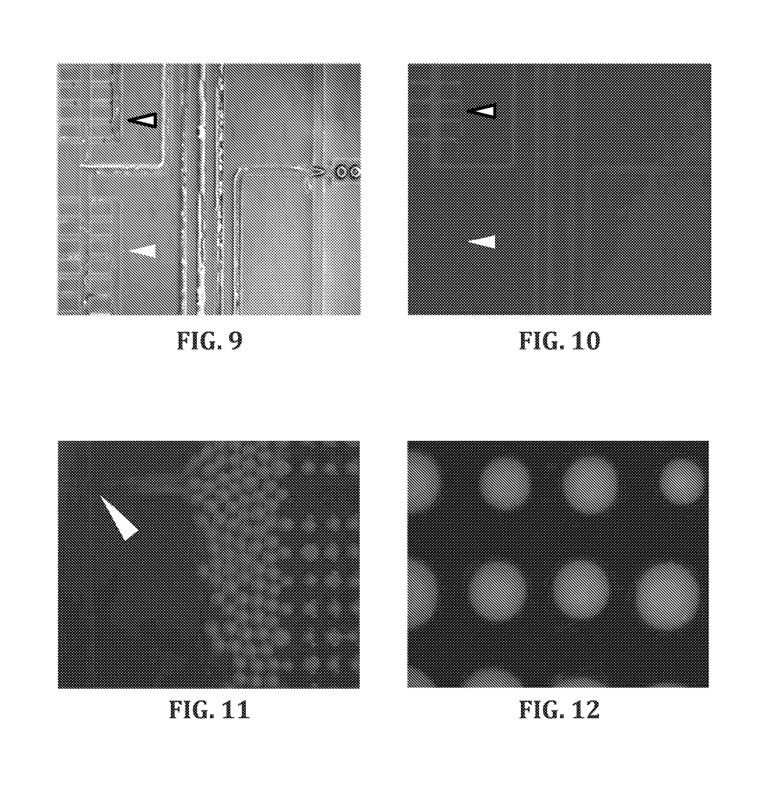

To further compare the present example system with standard ELISA, a calibration curve was constructed using four standard concentrations from a commercially available kit. Detection was possible for 0, 0.1, 0.5 and 1 UI ml⁻¹ of human anti-tetanus IgG using the present system. The equilibrium constant for tetanus toxoid-IgG antibody was previously reported to be about 10⁻⁷ to about 10⁻¹² M, implying very fast absorption reaction on the detection beads. The significant signal was at least about three standard deviations different from the background analyses. For each sample, at least 40 individual droplets with detecting beads (sensors) were analyzed (FIGS. 9-12). In particular, fast reagent mixing inside droplets is visualized on FIGS. 9-12. A stream of fluorescent reagents (anti-tetanus IgG conjugated with IgG Goat Anti-Human pAb, Alexa Fluor®488), indicated by the white arrows with black outlines on FIGS. 9 and 10, is rapidly mixed with the stream of non-florescent reagents (toxoid-conjugated microspheres), indicated by the white arrows with no outlines on FIGS. 9 and 10. The resulting droplets contain the mixed solutions, FIGS. 11 and 12. In one aspect, this rapid mixing can explain the increase in reaction kinetics rates previously observed in biochemical reactions in the flowing droplets.

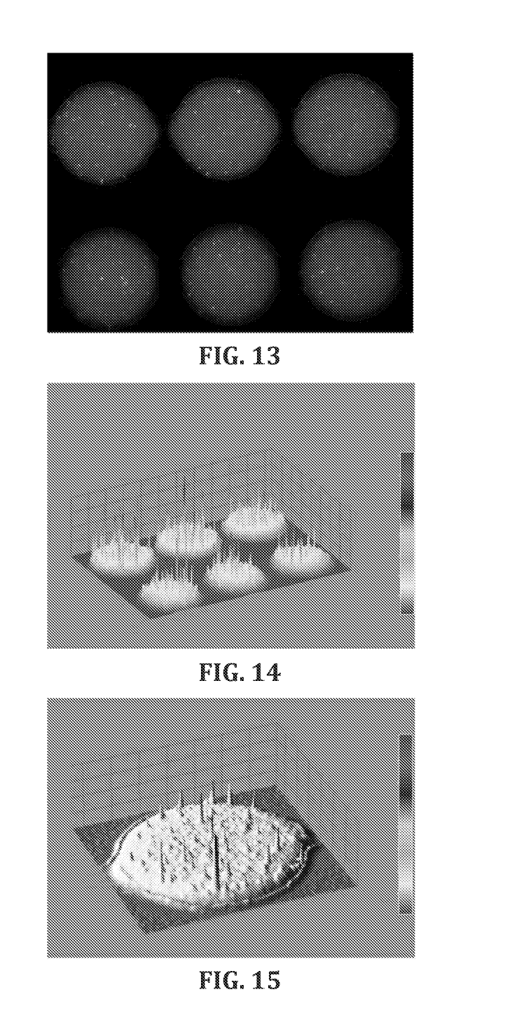

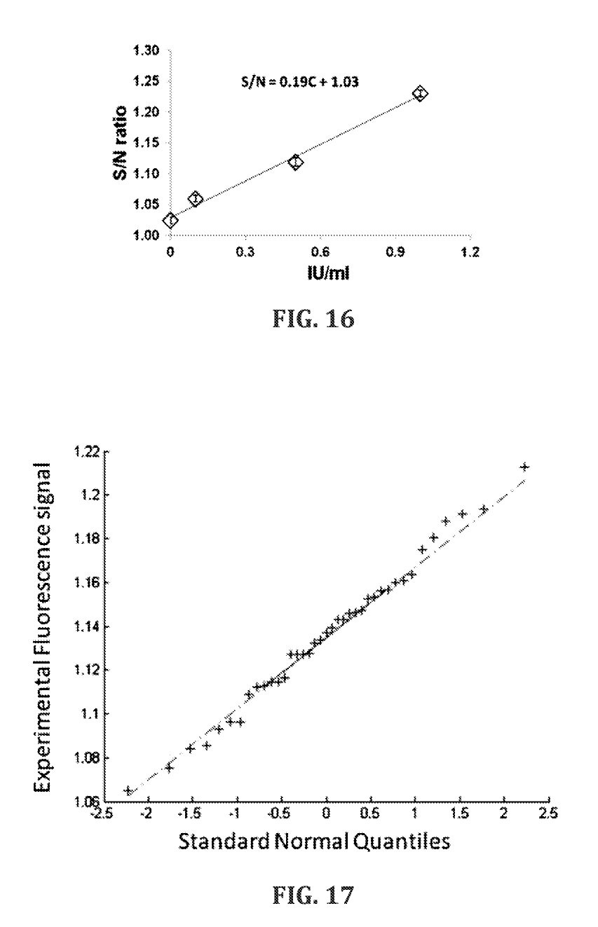

FIGS. 13, 14 and 15 show the images of the fluorescent signal on microsphere; the background is the unbounded fluorescently tagged secondary anti-human IgG antibody. The calibration curve of the droplet based immunosorbent assay appears in FIG. 16. For each calibration point, at least 40 individual droplets with detecting microspheres were analyzed. Detection of 0.1 IU mL-1 of human anti-tetanus IgG using picoliter droplet microsphere device was possible. Thus, the required sensitivity shown by ELISA for detecting anti-tetanus IgG in human sera was demonstrated. The signal to noise (S/N) ratio was calculated based on Eq. 23:

S/N=I/I₀ (Eq. 23)

where I is the average measured intensity from the bead center and I₀ is the average intensity from the background. Furthermore, using a mean square error method (R²=0.99) the equation that describes S/N ratio was calculated at steady-state on a detecting microsphere as a function of human anti-tetanus IgG concentration:

S/N=0.19C_{d}+1.03 (Eq. 24)

where C_{d} is the anti-tetanus IgG concentration in the sample.

The present detection platform was also validated using plasma samples collected from 500 adult immunized individuals. Using qqplot (FIG. 17), the measurements were shown to behave according to a Normal distribution with a mean of 1.14, a standard deviation of 0.03, a coefficient of variance of 0.03 and 5% confidence intervals of 1.0792 and 1.1925. Using Eq. 24 and multiplying the result by 2 in order to account for the dilution factor of sera inside the droplet, the average concentration of anti-tetanus antibodies was calculated to be about 1.12 IU mL⁻¹, which is in the range of previously reported concentrations of anti-tetanus IgG in human sera.

In one aspect, the total experiment time in the present system was less than 1 hour at room temperature (30 min for analyte-secondary antibody conjugation and 30 min for droplet formation and imaging). By contrast, a commercial assay can require more than 2.5 hours including washing steps and 37° C. incubation temperatures.

In one aspect, the present system and method can be applied to detect multiple analytes in the same sample by adding microspheres that bind other bio-analyte targets and different fluorophore tagged secondary antibodies. In combination with low cost optics and mechanics, the proposed microfluidic platform can further reduce the total cost of POC diagnostics. In another aspect, the incorporation of washing steps into the present design can further increase the sensitivity of the assay.

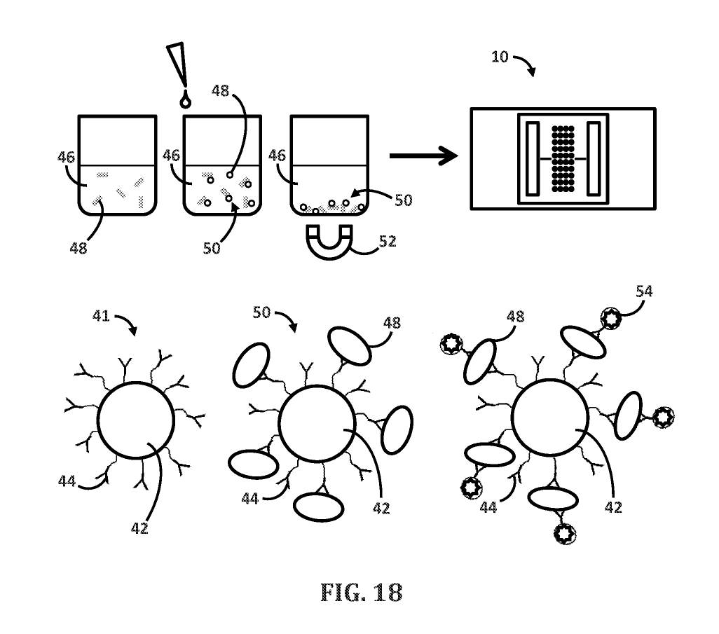

In another aspect, the present system and methods can include multiplexed bead-based E. coli capture and detection assays. The isolation of bacteria and detection are conducted utilizing a simple magnetic bead based immunoassay. Thus, no bacteria agar plate cultivation step is necessary to identify a presumptive positive sample. This approach saves considerable time and resources. FIG. 18 schematically illustrates one example approach in which a sensor complex 41 comprising magnetic beads 42 conjugated with anti-E. coli antibodies 44 were added to a water sample 46 containing bacterial cells 48. Within about 10 min, the sensor complex 41 captured the bacteria 48 (if any) from the water sample 46, thereby forming a plurality of sensor-analyte complexes 50. The sensor-analyte complexes 50 were then concentrated with a simple magnet 52, and a single immunoassay step was used to label the captured bacteria with a fluorescent antibody indicator 54 for subsequent detection. Detection protocols were integrated into a droplet microfluidic device 10 (FIG. 1) according to the present disclosure to reduce reagent volume and enhance reaction rates.

A POC system for multiplex analysis according to the present disclosure can include a droplet microfluidic device 10 for bacteria labeling, and a portable fluorescent optical system 60 for signal detection and sharing. Referring first to the droplet microfluidic device 10, to reduce reagent volumes and detection times, a picoliter droplet microfluidic chip was designed. The design of the microfluidic device 10 is shown in FIG. 1, and functions as described previously. Each droplet 22 in the array co-encapsulates fluorescently labeled anti-E. coli antibodies 54 with sensor-analyte complex 50 (if any), to generate a localized fluorescent signal for subsequent detection. The device 10 enables the generation and incubation of 103 droplets 22 with diameters of about 100 μm (about 520 pL). The advantages of this droplet-based array technique include the physical and chemical isolation of beads in droplets, and the rapid and efficient mixing of the reagents that occurs inside droplets providing fast reaction rates. Moreover, this picoliter microenvironment also enables gas exchange for bacterial viability if further studies are required.

To demonstrate the feasibility of the POC system for multiplex analysis, red florescent protein (RFP) and green florescent protein (GFP) expressing E. coli were co-encapsulated in the same droplet. Bead-based capture and subsequent encapsulation for detection of two different bacteria in the same microenvironment also enables a multiplex approach wherein several types of beads are conjugated with different antibodies that bind different target bacteria and different fluorescent tags.

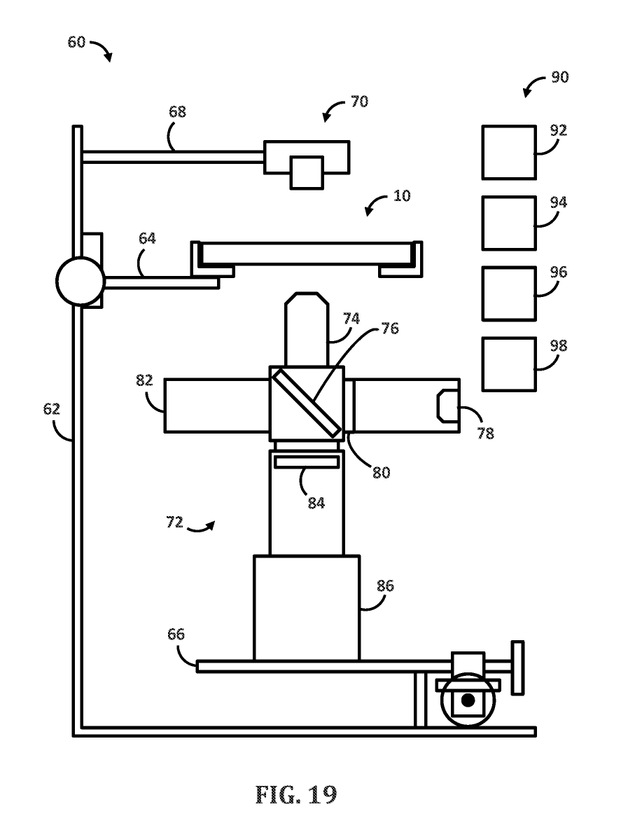

The schematic for the portable optical system 60 for fluorescent signal detection in the droplet microfluidic device is presented in FIG. 19. The system enables remote microscope control as well as simultaneous top and inverted image registration. The portable optical system 60 includes a scaffold 62 that supports a device housing 64, a robotic stage 66 and a camera mount 68. The device housing 64 is configured to retain a droplet microfluidic device 10 in a fixed position. However, in some embodiments, the position of the device housing 64 is adjustable. The camera mount 68 is configured to couple to a top camera 68 positioned above the device 10, which allows for bright field imaging of the entire field of the device 10. A bottom camera 72 is positioned beneath the device 10 for fluorescence imaging with 10× magnification. This combination of top camera 68 and bottom camera 72 allows for high-throughput droplet imaging.

With continued reference to FIG. 19, the bottom camera 72 includes a 10x microscope objective positioned above a dichroic filter 76. A light source 78 emits a beam that passes through an excitation filter 80 and is incident on the dichroic filter 76. At least a portion of the incident light is reflected off of the dichroic filter, passes through the microscope objective 74 and is incident on the detecting array 32 of the device 10. In the present example, the excitation wavelength is 470 nm. A light absorbing tube 82 opposes the light source 78 such that the dichroic filter 76 is positioned therebetween. An emission signal associated with droplets 22 in the device 10 can pass through the objective 74, through the dichroic filter 76 and then through an emission filter 84 positioned beneath dichroic filter 76. The emission signal is then detected by camera 86.

In one aspect, the robotic stage 66 is controlled by a motor 88 capable of repositioning the robotic stage 66 in a horizontal plane beneath the device housing 64. The robotic stage 66 is coupled to the bottom camera 72 in order to scan the array of droplets 22 within the device 10. In the present example embodiment, the robotic stage 66 has a microscope scanning range of 45 mm×45 mm and a resolution of 5 μm/step (10 mm/sec). Z-axis focus capabilities include 15 mm travel with 1 μm/step, at 2 mm/sec. The optical system 60 is controlled by a script-based programming language. In one aspect, the script can be automatically generated by a platform such as PR-PR (Linshiz et al., 2012. ACS Synthetic Biology 2: 216-222). A control system 90 for the optical system 60 can include an x86 PC with GPU 92, control electronics 94, system control software 96 and low signal sensitivity enhancement software 98.

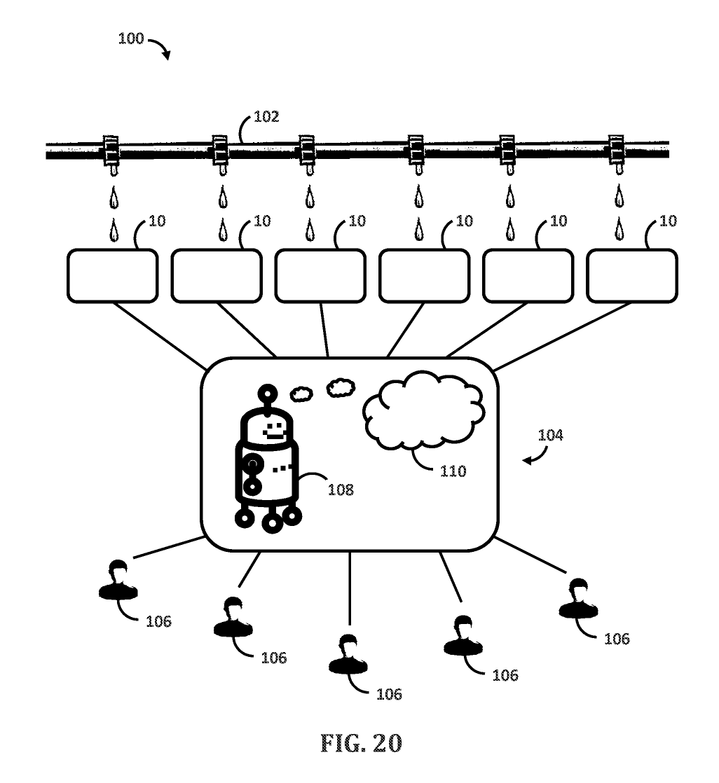

Referring to FIG. 20 a POC system 100 for multiplex analysis can include an analyte source 102 (e.g., water), a plurality of microfluidic devices 10, a cloud-based laboratory automation and data management system 104 and network of users 106 with shared access to the system 104. A biology-friendly high-level language for laboratory automation 108 was refined, to control the automated microscopy system 60 and enable prompt and easily adjusted changes to the experimental protocol. In an example script-based programming language, transfer of a material (e.g., a liquid) or system component (e.g., a robotic arm) is described by a Source, Destination, Quantity, and Method. In one example of the present system, the Source is the initial coordinates of the microscope stage (XY) and lens (Z), the Destination is the final target coordinates of interest, the Quantity is the number of pictures that should be taken, and the Method specifies imaging parameters: light, filters, and delay between image capture. An input script is provided to the automated microscope control (such as that presented in Example 3) and outputs a second script that can directly operate the automated microscope system 60. The script protocol and the resulting data for each experiment are stored in a local folder within the sensor and can be shared between users 106 via a cloud-based storage platform 110.

With respect to detection of RFP-expressing E. coli in drinking water, as a positive control, the ability of the present system to detect 150 CFU/mL of RFP-expressing E. coli in drinking water was tested according to the method illustrated in FIG. 18. The overall assay for E. coli detection is divided into three steps: enrichment, capture, and detection. For enrichment, 1 L of contaminated water sample was filtered and the filter with captured bacteria was incubated for 6 hours in the LB medium. For capturing, the enriched solution was mixed with Dynabeads® MAX anti-E. coli O157 for 10 min and separated by magnet.

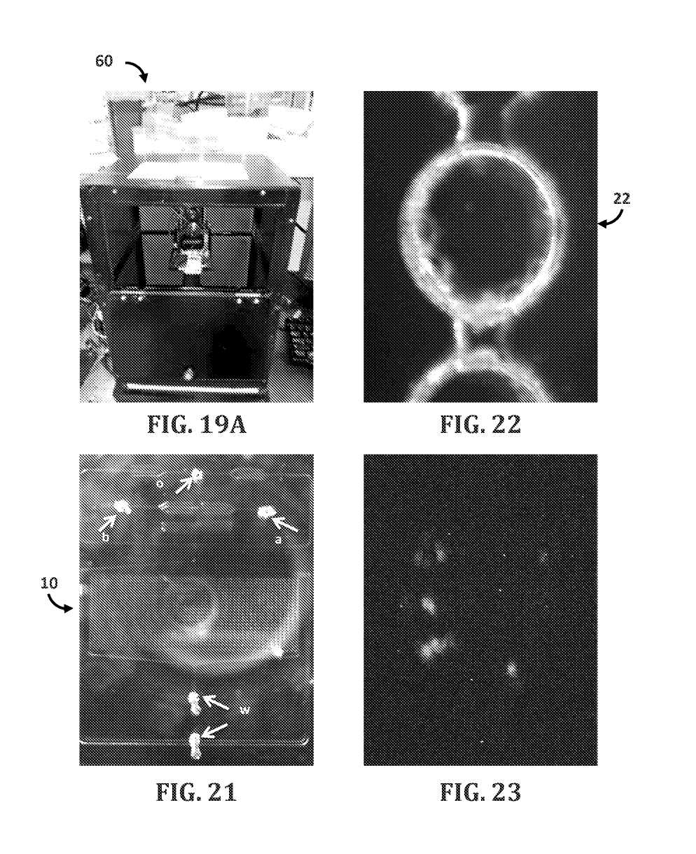

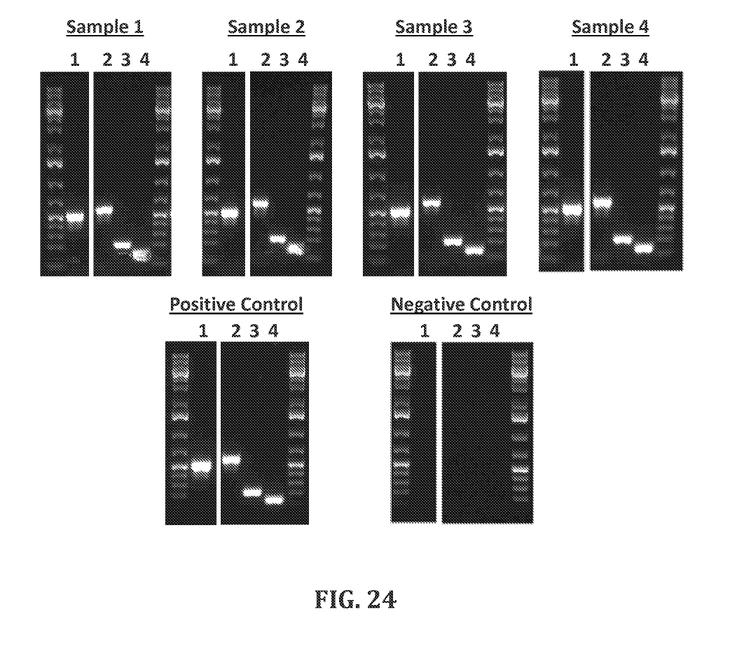

FIG. 21 shows the droplet-based microfluidic device 10 used to perform the immunoassay described in FIG. 18. For the detection step, beads conjugated to bacteria (sensor-analyte complex 41) captured from contaminated water sample 46 were co-encapsulated with secondary FITC fluorescently labeled anti-E. coli antibodies 54 in the droplet detecting array 32 of device 10 and incubated up to 1 hr in the detecting array 32 at room temperature. FIG. 22 shows a representative droplet, and FIG. 23 shows the green florescence signal (bright spots) detected in a single droplet containing E. coli capture on the bead and tagged by secondary FITC labeled antibodies. The presence of RFP expressing E. coli in water was confirmed by PCR (FIG. 24).

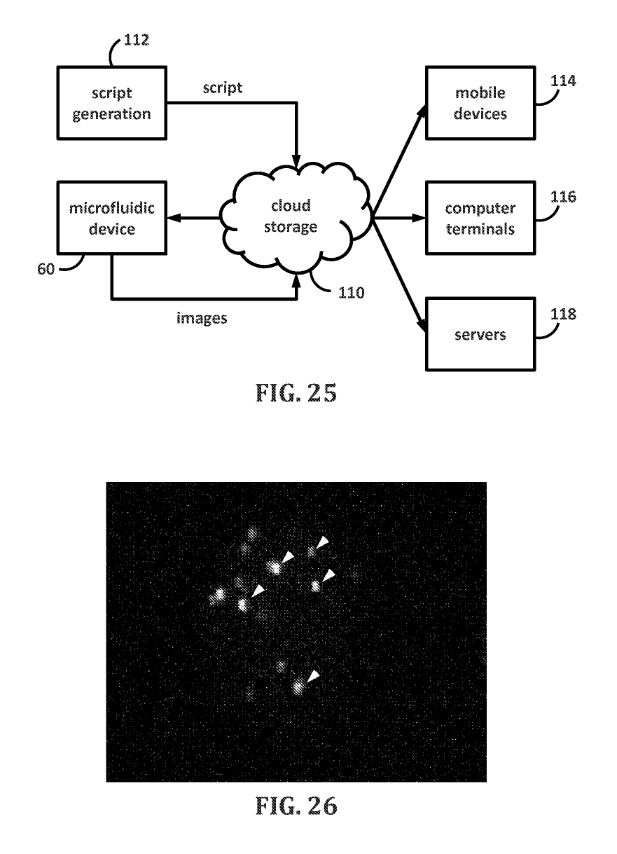

Next, the present system was tested for the detection of fecal E. coli in drinking water. Water was contaminated with rat feces and the droplet detection assay described above was performed. FIG. 25 presents an example method for the detection of fecal E. coli in water. In box 112, a script is generated by a user and uploaded to cloud storage 110. The script is then sent to the system 60 from cloud storage. The system 60 then uploads images and other data to cloud storage 110 from which the data can be shared with users 106 including mobile device 114, computer terminals 116 and servers 118.

FIG. 26 shows a representative resulting image, with fluorescence signal indicating E. coli contamination (white arrows). Successful detection by the present system was confirmed with PCR (FIG. 24), which clearly showed that the water samples were contaminated with E. coli. The results from the tests were uploaded to cloud-based data storage.

### Example 1

Droplet microfluidic flow focusing devices were fabricated using soft lithography. Negative photo resist SU-8 2100 (MicroChem, Newton, Mass.) was deposited onto clean silicon wafers to a thickness of 150 μm, and patterned by exposure to UV light through a transparency photomask (CAD/Art Services, Bandon, Oreg.). Sylgard 184 polydimethylsiloxane (PDMS) (Dow Corning, Midland, Mich.) was mixed with crosslinker (ratio 10:1), poured onto the photoresist patterns, degassed thoroughly and cured for 12 h at 75° C. The PDMS devices were peeled off the wafer and bonded to glass slides after oxygen-plasma activation of both surfaces. The microfluidic device was composed of two parts: a droplet forming nozzle (channel cross section 6.25*10⁻⁸ m², FIG. 1) and a 103 droplets storage array (channel cross section 3.13*10⁻⁷ m², FIG. 1). The multi-droplet array provided simultaneous measurement of multiple reactions, thus it decreased the standard error of the mean. On the day of the experiment, the microfluidic channels were treated with Pico-Sur™ 2 (Dolomite Microfluidics, UK) by filling the channels with the solution as received and then flushing them with air. This treatment was done to improve the wetting of the channels with mineral oil in the presence (1%, w/w) of the surfactant (span80). 1 mL syringes were used to load the fluids into the devices through Tygon Micro Bore PVC Tubing 100f, 0.010″ ID, 0.030″ OD, 0.010″ wall. The individual syringe pumps (FIG. 5) were used to control the flow rates of the oil and the reagents. The microspheres (0.5 mg mL⁻¹) conjugated with tetanus toxoid were injected through syringe pump 12; the analyte and fluorescently labeled secondary antibodies were injected through syringe pump 16. The oil phase was injected through syringe pump 23. To form droplets, the flow-rate-ratio of water-to-oil was adjusted to Q_{W}/Q₀=1.

In order to prepare the microsphere sensors, Clostridium difficile toxoid B protein was biotinylated with EZ-Link NHS-PEG4-biotin (Thermo Scientific, USA) according to the manufacture protocol. ProActive® Streptavidin Coated Microspheres (10 μm, Bang Laboratories Inc., USA) were conjugated with the biotinylated Toxoid B protein according to the manufacture protocol. Unbounded active sites were blocked with StarlingBlock™ (Thermo Scientific, USA) for one hour. The microspheres were washed with the Phosphate Buffered Saline (PBS), diluted to the final concentration 0.5 mg mL⁻¹, and stored at 4° C.

For immunosorbent detection of tetanus IgG, a calibration curve, with four standard concentrations (0, 0.1, 0.5 and 1 IU mL⁻¹) was constructed with the droplet microfluidics platform of the present disclosure. For the human plasma experiments, a pool of EDTA plasma bank, created from discarded specimens from about 500 healthy men and women was used. In these experiments, the human plasma was diluted by 2 using dilution buffer. In the first step, the analyzed IgG solution was mixed with IgG Goat Anti-Human pAb, Alexa Fluor®488 Conjugate secondary antibody (100:1 ratio) (Life Technologies, USA) and constantly mixed at the rotating disk for 15 min. In the second step, the two solutions were co-encapsulated within the aqueous droplet using the platform described herein. The first solution contained toxoid-conjugated microspheres at 0.5 mg mL⁻¹ (syringe pump 12, FIG. 1), and the second solution contained anti-tetanus IgG conjugated with IgG Goat Anti-Human pAb, Alexa Fluor®488 (syringe pump 16, FIG. 1). Flow rates were 20 μL min⁻¹ for the oil/surfactant mixture and 10 μL min⁻¹ for the assay solution. When the array was filled with droplets with encapsulated reagents, the flow was stopped. Each droplet had a size of about 100 μm and traveled about 2-5 mm on a chip to the specific location in the 1,000 well array. Florescence signal was detected from the microspheres up to 30 minutes post encapsulation.

With respect to image analyses, the detection antibodies, present in the droplets, were localized on microsphere captured analyte surface and generated a localized fluorescent detection signal. Fluorescence images were captured on a Zeiss 200 Axiovert microscope using an AxioCAM MRm digital camera and AxioVision 4.8 software. AlexaFluor488R fluorescence (excitation 488 nm/emission 525 nm) was detected to evaluate the reagent concentration. To calculate the positive detection signal, the signal to noise (S/N) ratio was calculated with Eq. 23, where I is the average measured intensity from the microsphere center and I₀ is the average intensity from the background inside the droplet. Image analysis software was used for image processing and a spreadsheet program with an external statistical package was used for statistical analyses.

### Example 2

Construction of the optics system included, in part, a custom made, motorized, dual view, computerized portable microscopy system designed for droplet microfluidic imaging (R&D Engineering Solutions, Netania, Israel). The dual view system was used for the simultaneous imaging of the whole chip (top view camera) and specific droplets (bottom view camera). The top view camera included a 1280×768 resolution, color sensor, auto/computer-controlled focus, manually configurable [83×50 mm-30×18 mm] field of view, 640×480 region of interest (ROI), and zoom functionality. The bottom view (microscope) camera was characterized by a 752×582 resolution, monochrome 8.6 μm×8.3 μm pixels sensor, and a 10× objective. A single 3W 468 nm light emitting diode (LED) was used for florescence excitation. A 41017—Endow GFP/EGFP bandpass fluorescence filter set (Chroma Inc., VT) was used for florescence detection. Top illumination was made by a single 30 mW white LED for chip observation and microscope camera positioning. An embedded x86 dual core computer with HDMI display port outputs (CompuLab, Israel) was used for the local control of the system. An embedded computer ran custom software, which allowed full control of the microscope, including XY position, focus, illumination, image acquisition and enhancement. A system can be controlled manually by the human operator via a standard PC console (keyboard, mouse and monitor). Alternatively, a system can be controlled programmatically via a program written in a coding language such as Python. Further improvements of the programmatic control aspect of the present system were made by leveraging PR-PR (Linshiz et al., 2012. ACS Synthetic Biology, 2(5):216-222), whereby a PR-PR microscope control script (Example 3) is translated into a script that can control the microscope system, as described above. Script deployment and image retrieval across distributed microscope systems was performed with a cloud-based storage service.

Bacterial strains and plasmids used included plasmids pFAB_SchPMK36GFP and pFAB_SchPMK36RFP. Each plasmid carried a kanamycin resistance marker. Plasmids were transformed into E. coli BW25113. Bacterial strains and plasmids, along with their associated information (e.g., annotated Genbank-format DNA sequence files), have been deposited in the public instance of the JBEI Registry (corresponding Part IDs JPUB_{—}001327-001329). For heat shock transformation, 1 μL pFAB_SchPMK36GFP or pFAB_SchPMK36RFP was mixed on ice with 40 μL chemically competent E. coli BW25113. The mixture was incubated on ice for 20 min, placed at 42° C. for 45 s, and then returned to ice. 200 μL SOC media was then added to each tube of transformed cells and incubated with agitation at 37° C. for 30 min. 100 μL of each transformation mixture was plated on solid LB media supplemented with 30 μg/mL kanamycin and then cultured at 37° C.

Construction of the microfluidic device for droplet generation was performed as described in Example 1 herein. Droplet microfluidics multiplex detection assays were carried out by first incubating E. coli expressing GFP or RFP were for 12 hours at 37° C. in LB media to 106 CFU/mL. Cells were then encapsulated into droplets. Fluorescence images were captured on a Zeiss 200 Axiovert microscope using an AxioCAM MRm digital camera and AxioVision 4.8 software at 20× magnification. Each experiment consisted of 4 replicates.

Detection of E. coli in water was performed by spiking 1 L of drinking water was with RFP-expressing E. coli to 150 CFU/mL. The spiked water was filtered through a 0.22 μm filter, and the filter was then used to inoculate 10 mL LB media, which was then incubated for 6 hr at 37° C. 20 μL of Dynabeads® MAX anti-E. coli O157 beads (Life Technologies, CA) were added to 1.5 mL of the incubation media and further incubated for 20 min on a rotating stage at room temperature (RT). Beads with captured bacteria were separated by magnet and resuspended in 400 μL of Phosphate Buffered Saline (PBS). The resuspended solution was co-encapsulated 500:1 with secondary antibody in droplet reactors inside the chip positioned on the robotic stage. After a further 1 hr of incubation at RT, images were taken from different locations on the chip. The objective position movements were controlled via script-based programming language, and the generated images were automatically uploaded to cloud-based storage. Each experiment consisted of 4 repeats.

For experiments involving drinking water contaminated with rat feces, fresh feces were collected from rat cages in the animal facility of Massachusetts General Hospital. 1.5 g feces was mechanically homogenized in 1 L of drinking water. The contaminated water was filtered twice through a 40 μm filter. The detection of E. coli in permeate was carried out using the present system and methods as described herein and by Real-Time PCR. Each experiment consisted of 4 replicates. Detection of RFP expressing and fecal origin E. coli by PCR was accomplished by selecting primer sets to detect E. coli in the prepared drinking water contaminated with rat feces. The primers targeted specific sequences from different loci in the E. coli genome: two primer sets for 16S rRNA, one for tuf, and one for uidA. For each primer set, four contaminated samples and a positive and a negative control were tested. Negative controls contained water only, and positive controls contained water supplemented with E. coli BW25113. After enrichment of the microbial population, 5 μL of enriched culture was added to 45 μL water. All samples were incubated 15 min at 98° C. and then diluted in additional 100 μL water. Each 30 μL PCR reaction contained 10 μL of the diluted cell lysate (as template), 10 μL of 3× qPCR master mix (water 3.3 μL, 5× Phusion HF 6 μL, dNTP 100 mM 0.25 μL, Phusion DNA Polymerase (NEB) 0.3 μL, SYBR® Green II 200× (Molecular Probes) 0.15 μL), and a pair of primers at 5 pmol each. PCR reactions were subjected to thermal cycling (3 min at 95° C., and then 30 cycles of 30 s at 95° C., 30 s at 58° C., and 30 s at 72° C., with a final hold step at 10° C.) in a Real-Time PCR System. The amplification curves were monitored and the PCR amplifications were stopped after most reactions plateaued. PCR fragments were analyzed using electrophoresis by running the PCR products in 1% agarose gels (FIG. 24). Each experiment consisted of 4 replicates.

PR-PR software is open-source software under the BSD license and is freely available from GitHub and is also available through its web interface on the public PR-PR webserver through the Joint Bioenergy Institute (JBEI).

### Example 3

An example script is provided as follows:

| NAME | Microscope_Experiment_1 | | | |
|---|---|---|---|---|
| LOCATION | Home | 5000, 6000, 2000 | | |
| LOCATION | Dest | 2000, 3000, 4000 | | |
| TRANSFER | Home | Dest(+5, +10, +20)*5 | 1 | lighton |
| TRANSFER | Home | +5, +10, +20*5 | 3 | lighton |
| TRANSFER | Dest | Dest(+5, +10, +20)*5 | 6 | lighton |

The present invention has been described in terms of one or more preferred embodiments, and it should be appreciated that many equivalents, alternatives, variations, and modifications, aside from those expressly stated, are possible and within the scope of the invention.

Each reference identified in the present application is herein incorporated by reference in its entirety.

While present inventive concepts have been described with reference to particular embodiments, those of ordinary skill in the art will appreciate that various substitutions and/or other alterations may be made to the embodiments without departing from the spirit of present inventive concepts. Accordingly, the foregoing description is meant to be exemplary, and does not limit the scope of present inventive concepts.

A number of examples have been described herein. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the present inventive concepts.

What is claimed is:

1. A method for a picoliter volume microfluidic assay comprising:
providing a first solution comprising a sensor;
preparing a sample comprising at least one analyte;
combining the sample with an indicator, thereby forming a second solution;
co-encapsulating the first solution and the second solution in a plurality of droplets with a microfluidic device;
incubating the plurality of droplets, thereby forming at least one complex comprising the sensor, at least one analyte and indicator; and
detecting the at least one complex,
wherein a primary signal associated with the at least one complex is distinguishable from a background signal associated with at least one of the sensor, at least one analyte and indicator individually.

2. The method of claim 1, wherein the volume of each of the plurality of droplets is less than about 10 μL.

3. The method of claim 2, wherein the volume of each of the plurality of droplets is less than about 1 μL.

4. The method of claim 3, wherein the volume of each of the plurality of droplets is about 500 pL.

5. The method of claim 1, wherein the size of each of the plurality of droplets is about 100 μm.

6. The method of claim 1, wherein the step of co-encapsulating further comprises passing the first and second solutions through a nozzle of the microfluidic device.

7. The method of claim 1, further comprising arraying the plurality of droplets into a set of wells within the microfluidic device.

8. The method of claim 1, wherein the sensor comprises a microsphere.

9. The method of claim 1, wherein the indicator comprises fluorophore.

10. The method of claim 1, wherein the at least one analyte comprises a biological sample.

11. A system for performing a picoliter volume microfluidic assay comprising:
a microfluidic device comprising:
a first solution source;
a second solution source;
a nozzle in communication with the first and second solution sources, the nozzle configured for co-encapsulating the first solution and the second solution in a plurality of droplets within the microfluidic device;
an array of wells in communication with the nozzle, the array of wells configured to receive the plurality of droplets; and
a detector configured to detect a primary signal from at least one complex within at least one of the plurality of droplets,
wherein the at least one complex comprises a sensor, at least one analyte and an indicator, and
wherein a primary signal associated with the at least one complex is distinguishable from a background signal associated with at least one of the sensor, at least one analyte and indicator individually.

12. The system of claim 11, wherein the volume of each of the plurality of droplets is less than about 10 μL.

13. The system of claim 12, wherein the volume of each of the plurality of droplets is less than about 1 μL.

14. The system of claim 13, wherein the volume of each of the plurality of droplets is about 500 pL.

15. The system of claim 11, wherein the size of each of the plurality of droplets is about 100 μm.

16. The system of claim 11, wherein the detector is a fluorescence microscope.

17. The system of claim 11, wherein the sensor comprises a microsphere.

18. The system of claim 11, wherein the indicator comprises fluorophore.

19. The system of claim 11, wherein the at least one analyte comprises a biological sample.

20. The system of claim 11, wherein the biological sample comprises an antibody.
